(19) United States
(12) Patent Application Publication
(10) Pub. No.: US 2024/0037736 A1
(43) Pub. Date: Feb. 1, 2024

(54) INSPECTION ASSISTANCE DEVICE, INSPECTION ASSISTANCE METHOD, AND RECORDING MEDIUM

(71) Applicant: NEC Corporation, Minato-ku, Tokyo (JP)

(72) Inventors: Ayaka Amakawa, Tokyo (JP); Maki Sano, Tokyo (JP); Yatin Joshi, Tokyo (JP)

(73) Assignee: NEC Corporation, Minato-ku, Tokyo (JP)

(21) Appl. No.: 18/267,585

(22) PCT Filed: Mar. 25, 2021
(86) PCT No.: PCT/JP2021/012496

§ 371 Date: Jun. 15, 2023

(51) Int. Cl. G06T 7/00; G06V 20/69; G16B 20/00

(52) U.S. Cl. CPC G06T 7/0012; G06V 20/695; G16B 20/00; G06V 2201/03; G06T 2207/20084; G06T 2207/20081; G06T 2207/30096; G16H 30/20

(57) ABSTRACT

Provided is a technique that suggests a testing region suited to dissection in genetic testing of somatic cells. An acquisition unit (11) acquires image data of a pathological specimen; an estimation unit (12) estimates the content ratio of tumor cells in each of individual regions in a focus region in the image data of the pathological specimen; a determination unit (13) determines a testing region in the focus region on the basis of the content ratio of tumor cells in each of the individual regions in the focus region; and an output unit (14) outputs information indicating the testing region.

### TECHNICAL FIELD

The present invention relates to an inspection assistance device, an inspection assistance method, and a recording medium, and more particularly, to an inspection assistance device, an inspection assistance method, and a recording medium that assist an inspection of a tumor using a pathology specimen.

### BACKGROUND ART

In order to acquire a cell population to be subjected to a genetic test of somatic cells, a device in which a laser irradiation device is connected to a microscope is used. In one method, the sliced tissue is attached to a dedicated slide and stained, and then a laser is radiated along the contour of the test region while observing a section of the tissue with a microscope. As a result, the cell population in the test region can be separated from the tissue and collected. This is an example of a technique called die section (dissociation).

In a related technology, diagnosis and diagnosis assistance are performed using a digitized pathological image. For example, in the related technique described in PTL 1, a pathological tissue is automatically identified using a pathological image with a high magnification and a pathological image with a low magnification. This technique is an example of digital pathology.

In the technique described in PTL 2, description is made in which a state of a cell or a tissue in a pathological image is identified using a training model machine trained with a large number of samples of the pathological image, and the identification result is visualized.

### CITATION LIST

### Patent Literature

### PTL 1: JP 2010-203949 A

### PTL 2: JP 2018-044806 A

### SUMMARY OF INVENTION

### Technical Problem

For an accurate genetic test, it is required to select a test region having a high content rate of tumor cells from the tissue. However, it is difficult for a pathologist to appropriately determine a test region having a high content rate of tumor cells. An unfamiliar pathologist may not be able to manually and accurately dissect (dissociate) the cell population in the test region.

The present invention has been made in view of the above problems, and an object of the present invention is to provide a technique for proposing a test region suitable for a die section in a genetic test of somatic cells.

### Solution to Problem

An inspection assistance device according to an aspect of the present invention, an inspection assistance device includes an acquisition means configured to acquire image data of a pathology specimen, an estimation means configured to estimate a tumor cell content rate for each unit region in a region of interest in image data of the pathology specimen, a determination means configured to determine a test region in the region of interest based on the tumor cell content rate in the region of interest, and an output means configured to output information indicating the test region.

An inspection assistance method according to an aspect of the present invention includes acquiring image data of a pathology specimen, estimating a tumor cell content rate for each unit region in a region of interest in image data of the pathology specimen, determining a test region in the region of interest based on the tumor cell content rate in the region of interest, and outputting information indicating the test region.

A non-transitory storage medium according to an aspect of the present invention records a program for causing a computer to execute the steps of acquiring image data of a pathology specimen, estimating a tumor cell content rate for each unit region in a region of interest in image data of the pathology specimen, determining a test region in the region of interest based on the tumor cell content rate in the region of interest, and outputting information indicating the test region.

### Advantageous Effects of Invention

According to an aspect of the present invention, it is possible to propose a test region suitable for a die section in a genetic test of somatic cells.

### BRIEF DESCRIPTION OF DRAWINGS

FIG. 1 is a diagram illustrating an example of transmission and reception of data in a system including a terminal of a pathologist and a server.

FIG. 2 is a diagram schematically illustrating an example of image data of a pathology specimen.

FIG. 3 is an example illustrating a screen of a terminal of a pathologist, in which information indicating a tumor cell content rate for each unit region is added on one piece of image data of a pathology specimen.

FIG. 4 illustrates a test region in a region of interest by a line on image data of the pathology specimen illustrated in FIG. 3.

FIG. 5 is a view schematically illustrating a unit region containing normal cells and tumor cells.

FIG. 6 is a block diagram illustrating a configuration of an inspection assistance device according to the first example embodiment.

FIG. 7 is a flowchart illustrating an operation of the inspection assistance device according to the first example embodiment.

FIG. 8 is a diagram illustrating an example of a hardware configuration of the inspection assistance device according to the first example embodiment.

### EXAMPLE EMBODIMENT

Example embodiments of the present invention will be described with reference to the drawings.

### System 1

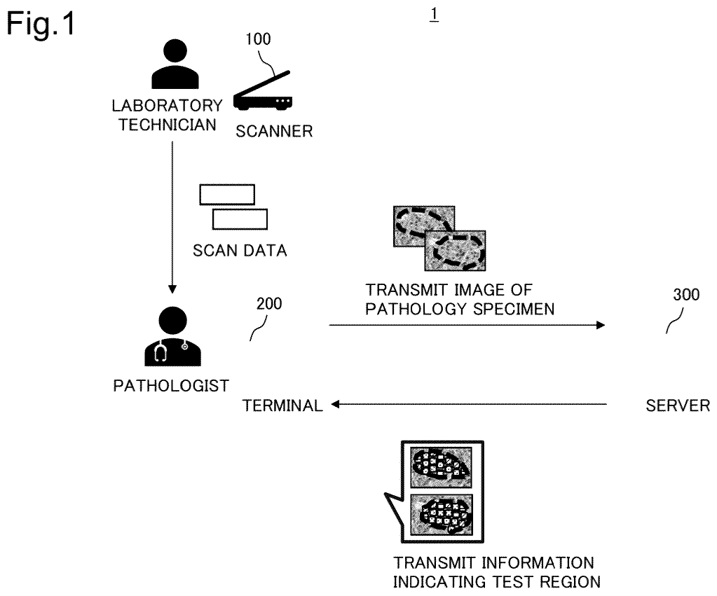

An example of a configuration of a system 1 to which an inspection assistance device 10 according to a first example embodiment described later is applied will be described with reference to FIG. 1. FIG. 1 is a diagram schematically illustrating an example of a configuration of a system.

As illustrated in FIG. 1, the system 1 includes a scanner 100 of a laboratory technician, a terminal 200 of a pathologist, and a server 300.

The laboratory technician creates a pathology specimen of cellular tissue to be subjected to a genetic test. The laboratory technician creates scan data of the pathology specimen using the scanner 100. The laboratory technician transmits the created scan data of the pathology specimen to the terminal 200 of the pathologist.

The pathologist creates image data of the pathology specimen to be transmitted to the server 300 by processing the scan data of the pathology specimen received by the terminal 200. For example, the pathologist determines a region of interest that is considered to have a high tumor cell content rate.

The pathologist annotates the region of interest. For example, a pathologist may add markings to a region of interest that is considered to have a high tumor cell content rate using general image editing software. For example, the pathologist inputs dots or lines in such a way as to surround the region of interest on the image data of the pathology specimen using general image editing software operating on the terminal 200. Alternatively, the pathologist may acquire the pathology specimen itself created by the laboratory technician, and after drawing dots or lines on the pathology specimen with a magic pen or the like, may scan the pathology specimen using the scanner 100. The terminal 200 of the pathologist transmits the image data of the pathology specimen created in this manner to the server 300.

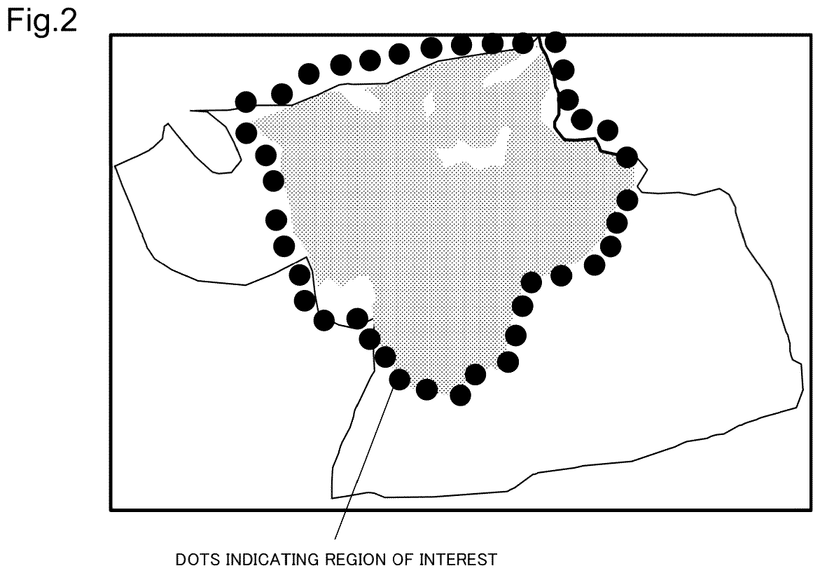

FIG. 2 schematically illustrates an example of image data of a pathology specimen. In the image data of the pathology specimen illustrated in FIG. 2, a dot indicating the region of interest is added to a dark color portion. The case where the pathologist designates the region of interest is described. However, the region of interest does not necessarily need to be designated. In a case where the region of interest is not designated, in the following description, the “region of interest” is read as the entire image data of the pathology specimen.

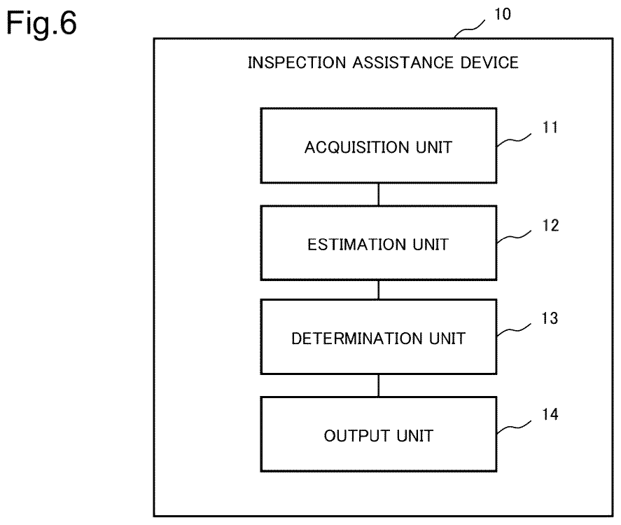

The server 300 includes an inspection assistance device 10 (FIG. 6) according to the first example embodiment described later. As described in detail in the first example embodiment, the inspection assistance device determines the test region in the region of interest by analyzing the image data of the pathology specimen.

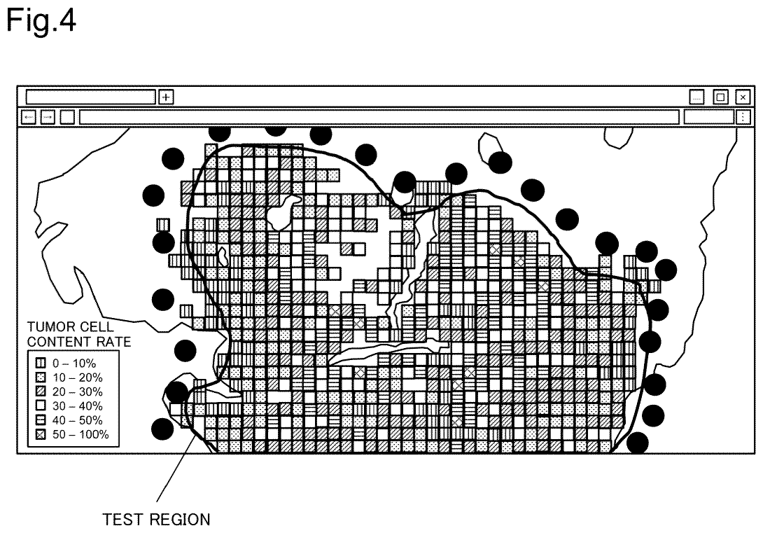

The server 300 transmits information indicating the determined test region to the terminal 200 of the pathologist. For example, the server 300 may add a line indicating the test region proposed to the pathologist on the image data of the pathology specimen (FIG. 4).

The terminal 200 of the pathologist displays the information indicating the test region received from the server 300. A detailed configuration and operation of the inspection assistance device 10 included in the server 300 will be described in the first example embodiment.

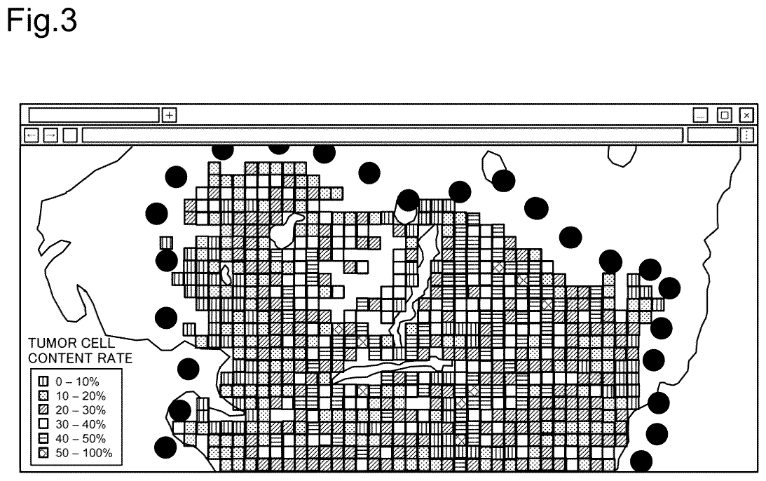

FIG. 3 is an example illustrating a screen of the terminal 200 of the pathologist. FIG. 3 illustrates the tumor cell content rate for each unit region in the pathology specimen. More specifically, in FIG. 3, the magnitude of the tumor cell content rate for each unit region in the region of interest is represented by a pattern on one piece of image data of the pathology specimen. In FIG. 3, the tumor cell content rate for each unit region is classified by 10%. The unit region is a region in one rectangle when one piece of image data of the pathology specimen is divided into rectangles having a certain size. The unit region is sufficiently larger than the size of one cell. Therefore, a large number of cells and/or tumor cells are present in the unit region. Hereinafter, the ratio of tumor cells in one unit region of interest is referred to as a tumor cell content rate. That is, the tumor cell content rate is a ratio of the number of tumor cells to the total number of cells and tumor cells contained in one unit region of interest.

In FIG. 4, the test region in the region of interest is illustrated by a line on the image data of the pathology specimen illustrated in FIG. 3. The line of the test region in the image data of the pathological image illustrated in FIG. 4 is an example of information indicating the test region described above.

### Description of Calculation Method of Tumor Cell Content Rate

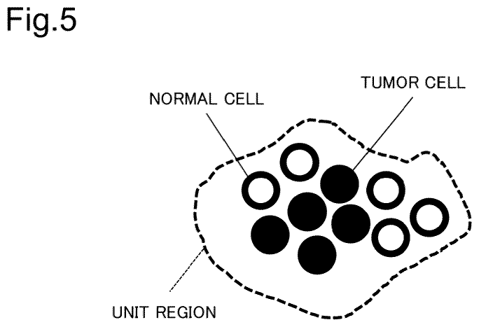

With reference to FIG. 5, a method of calculating the tumor cell content rate will be described. The tumor cell content rate is a ratio of tumor cells in a unit region. FIG. 5 schematically illustrates normal cells and tumor cells within a unit region. In the example illustrated in FIG. 5, since five of the seven cells contained in the unit region are tumor cells, the tumor cell content rate is about 71% (=5/7×100).

### First Example Embodiment

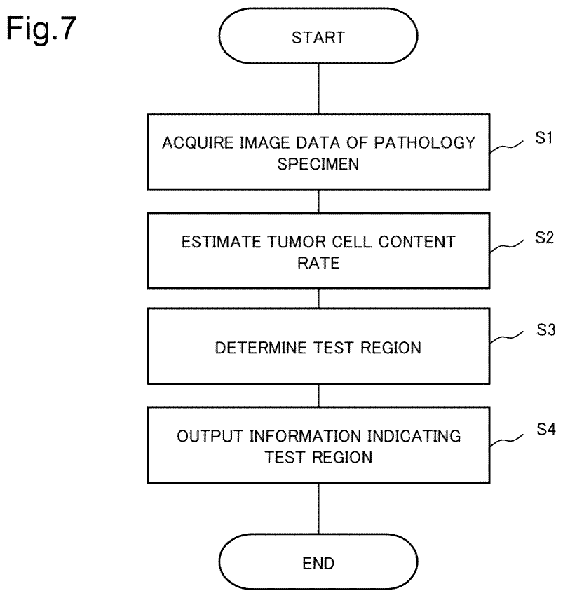

The first example embodiment will be described with reference to FIGS. 6 to 7. In the first present example embodiment, the “tumor cell content rate” means the ratio of tumor cells to the entire cells included in the region of interest (unit region).

### Inspection Assistance Device 10

With reference to FIG. 6, components included in the inspection assistance device 10 according to the first present example embodiment will be described. FIG. 6 is a block diagram illustrating a configuration of the inspection assistance device 10. As illustrated in FIG. 6, the inspection assistance device 10 includes an acquisition unit 11, an estimation unit 12, a determination unit 13, and an output unit 14.

The acquisition unit 11 acquires image data of the pathology specimen. The acquisition unit 11 is an example of an acquisition means. In an example, the acquisition unit 11 acquires the image data of the pathology specimen transmitted from the terminal 200 (FIG. 1) of the pathologist to the server 300 (FIG. 1). The acquisition unit 11 outputs the acquired image data of the pathology specimen to the estimation unit 12.

The estimation unit 12 estimates the tumor cell content rate for each unit region in the region of interest in the image data of the pathology specimen. The region of interest is a region determined by the pathologist to have a content rate of tumor cells higher than that of normal cells on the image data of the pathology specimen. In an example, the estimation unit 12 identifies the region of interest based on dots (FIG. 2) on the image data added by the image editing software operating on the terminal 200 of the pathologist. In this case, the estimation unit 12 may set a region surrounded by a line formed by connecting adjacent (that is, nearest) dots with a line as the region of interest.

Furthermore, the estimation unit 12 estimates the tumor cell content rate for each unit region in the region of interest in the image data using an identifier that has been machine trained on the characteristics of the cells. For example, the estimation unit 12 estimates the tumor cell content rate in each unit region using a neural network that was trained on a model such as a tumor cell. The estimation unit 12 may estimate the tumor cell content rate using the related technology described in PTL 2.

The estimation unit 12 outputs information indicating the tumor cell content rate for each unit region in the region of interest in the image data to the determination unit 13.

The determination unit 13 determines the test region in the region of interest based on the tumor cell content rate for each unit region in the region of interest. The determination unit 13 is an example of a determination means.

In an example, the determination unit 13 determines the test region in the region of interest in such a way that the total average of the tumor cell content rates of all the unit regions included in the test region is equal to or greater than the first threshold value. In one specific example, the determination unit 13 determines the test region in the region of interest in such a way that the total average of the tumor cell content rates of all the unit regions included in the test region is 30% or more. However, the first threshold value may be determined to be any value

In another example, the determination unit 13 determines the test region in the region of interest in such a way that the total average of the indices of all the unit regions included in the test region is equal to or greater than the second threshold value. The “index” represents the magnitude of the tumor cell content rate of the unit region. An example of the index will be described with reference to FIG. 3.

In the example illustrated in FIG. 3, the unit regions are distinguished by ranks according to the magnitude of the tumor cell content rate. Specifically, the unit region is divided into six ranks of “0 to 10%”, “10 to 20%”, “20 to 30%”, “30 to 40%”, “40 to 50%”, and “50 to 100%”. “to %” refers to a tumor cell content rate. In this example, the rank according to the tumor cell content rate of the unit region corresponds to the index of the unit region.

In one specific example, the determination unit 13 may determine the test region in the region of interest in such a way that the total average of the indices of all the unit regions included in the test region is equal to or greater than 4 of 6 levels. The second threshold value may be determined to be any value independently of the first threshold value.

In a modification, the determination unit 13 determines the test region based on at least one of the second condition related to the size of the area of the test region and the third condition related to the shape of the contour of the test region in addition to the first condition related to the tumor cell content rate in the region of interest.

For example, the second condition is that the area of the test region exceeds a first lower limit value. The third condition is that the contour of the test region is a smooth curve. However, the second condition and the third condition are not limited thereto.

The determination unit 13 outputs information indicating the test region in the region of interest to the output unit 14.

The output unit 14 outputs information indicating the test region. The output unit 14 is an example of an output means.

In an example, the output unit 14 receives information indicating the test region in the region of interest from the determination unit 13.

Then, the output unit 14 outputs information indicating the test region to the terminal 200 (FIG. 1) of the pathologist via the local network or the Internet. In an example, the output unit 14 outputs the image data of the pathology specimen indicating the tumor cell content rate for each unit region in the region of interest to the terminal 200 of the pathologist. The output unit 14 adds a line indicating the test region on the output image data (FIG. 4). In this example, a line indicating the test region added on the image data of the pathology specimen corresponds to the information indicating the test region. Alternatively, the output unit 14 may transmit information indicating the test region to the terminal 200 of the pathologist via a wireless or wired network, and display the image of the test region illustrated in FIG. 4 on the terminal 200.

### Operation of Inspection Assistance Device 10

The operation of the inspection assistance device 10 according to the first present example embodiment will be described with reference to FIG. 7. FIG. 7 is a flowchart illustrating a flow of execution number processing by each unit of the inspection assistance device 10.

As illustrated in FIG. 7, the acquisition unit 11 acquires image data (FIG. 2) of the pathology specimen (S1). The acquisition unit 11 outputs the image data of the pathology specimen to the estimation unit 12.

The estimation unit 12 estimates the tumor cell content rate for each unit region in the region of interest in the image data of the pathology specimen (S2). The estimation unit 12 outputs information indicating the tumor cell content rate in the region of interest to the determination unit 13.

The determination unit 13 determines the test region in the region of interest based on the tumor cell content rate (FIG. 3) for each unit region in the region of interest (S3). The determination unit 13 outputs information indicating the test region to the output unit 14.

The output unit 14 outputs information indicating the test region (S4). The output unit 14 displays the information indicating the test region on the screen of the terminal of the pathologist. In an example, as illustrated in FIG. 4, the output unit 14 displays, on the terminal of the pathologist, a screen in which the test region is illustrated by a line in the image data of the pathology specimen.

Thus, the operation of the inspection assistance device 10 ends.

### Effects of Present Example Embodiment

According to the configuration of the present example embodiment, the acquisition unit 11 acquires image data of the pathology specimen. The estimation unit 12 estimates the tumor cell content rate for each unit region in the region of interest in the image data of the pathology specimen. The determination unit 13 determines the test region in the region of interest based on the tumor cell content rate for each unit region in the region of interest. The output unit 14 outputs information indicating the test region. The test region to be output is determined based on the tumor cell content rate for each unit region in the region of interest. Generally, it can be said that the higher the tumor cell content rate in the test region, the more the test region is suitable for a die section. Therefore, in a genetic test of somatic cells, a test region suitable for a die section can be proposed.

### Hardware Configuration

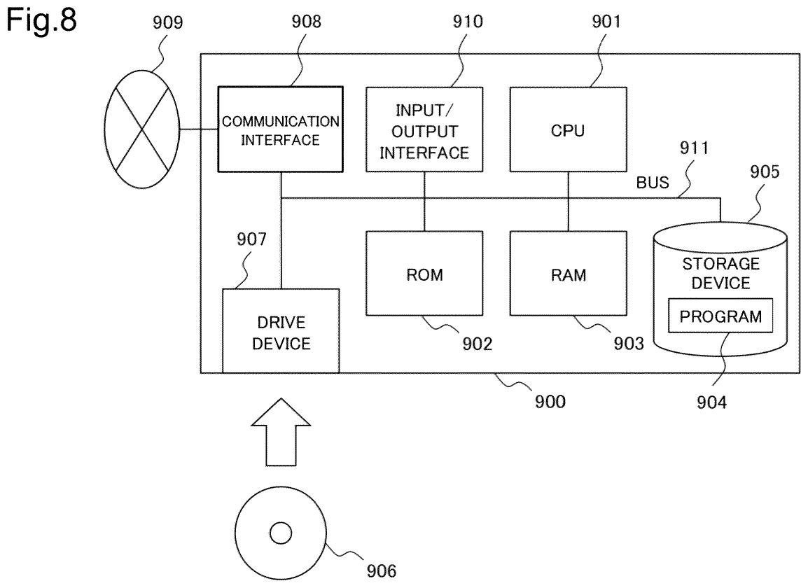

Each component of the inspection assistance device 10 described in the first example embodiment indicates a block of a functional unit. Some or all of these components are implemented by an information processing device 900 as illustrated in FIG. 8, for example. FIG. 8 is a block diagram illustrating an example of a hardware configuration of the information processing device 900.

As illustrated in FIG. 8, the information processing device 900 includes the following configuration as an example.
central processing unit (CPU) 901
read only memory (ROM) 902
random access memory (RAM) 903
program 904 loaded into RAM 903
storage device 905 storing program 904
drive device 907 that reads and writes recording medium 906
communication interface 908 connected to a communication network 909
input/output interface 910 for inputting/outputting data
bus 911 connecting respective components

Each component of the inspection assistance device 10 described in the first example embodiment is achieved by the CPU 901 reading and executing the program 904 for achieving these functions. The program 904 for achieving the function of each component is stored in the storage device 905 or the ROM 902 in advance, for example, and the CPU 901 loads the program into the RAM 903 and executes the program as necessary. The program 904 may be supplied to the CPU 901 via the communication network 909, or may be stored in advance in the recording medium 906, and the drive device 907 may read the program and supply the program to the CPU 901.

According to the above configuration, the inspection assistance device 10 described in the first example embodiment is achieved as hardware. Therefore, effects similar to the effects described in the above example embodiment can be obtained.

Although the present invention is described with reference to the example embodiments (and examples), the present invention is not limited to the above example embodiments (and examples). Various modifications that can be understood by those skilled in the art can be made to the configurations and details of the above example embodiments (and examples) within the scope of the present invention.

Some or all of the above example embodiments may be described as the following Supplementary Notes, but are not limited to the following.

### Supplementary Note

### Supplementary Note 1

An inspection assistance device including

an acquisition means configured to acquire image data of a pathology specimen,

an estimation means configured to estimate a tumor cell content rate for each unit region in a region of interest in image data of the pathology specimen,

a determination means configured to determine a test region in the region of interest based on the tumor cell content rate in the region of interest, and

an output means configured to output information indicating the test region.

### Supplementary Note 2

The inspection assistance device according to Supplementary Note 1, wherein

the determination means

determines the test region based on a first condition related to a tumor cell content rate in the test region.

### Supplementary Note 3

The inspection assistance device according to Supplementary Note 2, wherein

the determination means

determine the test region in such a way that an average of indices based on a tumor cell content rate in the test region exceeds a first threshold value according to the first condition.

### Supplementary Note 4

The inspection assistance device according to Supplementary Note 2 or 3, wherein

the determination means

determines the test region based on at least one of a second condition related to a size of an area of the test region and a third condition related to a shape of a contour of the test region in addition to the first condition.

### Supplementary Note 5

The inspection assistance device according to any one of Supplementary Notes 1 to 4, wherein

the estimation means

calculates an index based on a tumor cell content rate in the region of interest.

### Supplementary Note 6

The inspection assistance device according to any one of Supplementary Notes 1 to 5, wherein

the output means

displays a tumor cell content rate for each unit region in the region of interest on the image.

### Supplementary Note 7

The inspection assistance device according to any one of Supplementary Notes 1 to 6, wherein

the acquisition means acquires image data of the pathology specimen to which information indicating the region of interest is given, and wherein

the output means outputs information indicating the region of interest together with information indicating the test region.

### Supplementary Note 8

The inspection assistance device according to any one of Supplementary Notes 1 to 7, wherein

the estimation means estimates a tumor cell content rate for each unit region in the region of interest using a neural network that was trained on a tumor model.

### Supplementary Note 9

The inspection assistance device according to Supplementary Note 7, wherein

the information indicating the region of interest is given with an annotation.

### Supplementary Note 10

The inspection assistance device according to Supplementary Note 4, wherein

the second condition is that an area of the test region exceeds a second threshold value.

### Supplementary Note 11

The inspection assistance device according to Supplementary Note 4, wherein

the third condition is that a contour of the test region is a smooth curve.

### Supplementary Note 12

An inspection assistance method including

acquiring image data of a pathology specimen,

estimating a tumor cell content rate for each unit region in a region of interest in image data of the pathology specimen,

determining a test region in the region of interest based on the tumor cell content rate in the region of interest, and

outputting information indicating the test region.

### Supplementary Note 13

A non-transitory recording medium recording a program for causing a computer to execute the steps of

acquiring image data of a pathology specimen,

estimating a tumor cell content rate for each unit region in a region of interest in image data of the pathology specimen,

determining a test region in the region of interest based on the tumor cell content rate in the region of interest, and

outputting information indicating the test region.

### REFERENCE SIGNS LIST

10 inspection assistance device
11 acquisition unit
12 estimation unit
13 determination unit
14 output unit

What is claimed is:

1. An inspection assistance device comprising:
a memory configured to store instructions; and
at least one processor configured to execute the instructions to perform:
acquiring image data of a pathology specimen;
estimating a tumor cell content rate for each unit region in a region of interest in image data of the pathology specimen;
determining a test region in the region of interest based on the tumor cell content rate in the region of interest; and
outputting information indicating the test region.

2. The inspection assistance device according to claim 1, wherein
the at least one processor is configured to execute the instructions to perform:
determining the test region based on a first condition related to a tumor cell content rate in the test region.

3. The inspection assistance device according to claim 2, wherein
the at least one processor is configured to execute the instructions to perform:
determining the test region in such a way that an average of indices based on a tumor cell content rate in the test region exceeds a first threshold value according to the first condition.

4. The inspection assistance device according to claim 2, wherein
the at least one processor is configured to execute the instructions to perform:
determining the test region based on at least one of a second condition related to a size of an area of the test region and a third condition related to a shape of a contour of the test region in addition to the first condition.

5. The inspection assistance device according to claim 14, wherein
the at least one processor is configured to execute the instructions to perform:
calculating an index based on a tumor cell content rate in the region of interest.

6. The inspection assistance device according to claim 1, wherein
the at least one processor is configured to execute the instructions to perform:
displaying a tumor cell content rate for each unit region in the region of interest on the image.

7. The inspection assistance device according to claim 1, wherein
the at least one processor is configured to execute the instructions to perform:
acquiring image data of the pathology specimen to which information indicating the region of interest is given, and
outputting information indicating the region of interest together with information indicating the test region.

8. The inspection assistance device according to claim 1, wherein
the at least one processor is configured to execute the instructions to perform:
estimating a tumor cell content rate for each unit region in the region of interest using a neural network that was trained on a tumor model.

9. The inspection assistance device according to claim 7, wherein
the information indicating the region of interest is given with an annotation.

10. The inspection assistance device according to claim 4, wherein
the second condition is that an area of the test region exceeds a first lower limit value.

11. The inspection assistance device according to claim 4, wherein
the third condition is that a contour of the test region is a smooth curve.

12. An inspection assistance method comprising:
acquiring image data of a pathology specimen;
estimating a tumor cell content rate for each unit region in a region of interest in image data of the pathology specimen;
determining a test region in the region of interest based on the tumor cell content rate in the region of interest; and
outputting information indicating the test region.

13. A non-transitory recording medium recording a program for causing a computer to execute the steps of:
acquiring image data of a pathology specimen;
estimating a tumor cell content rate for each unit region in a region of interest in image data of the pathology specimen;
determining a test region in the region of interest based on the tumor cell content rate in the region of interest; and
outputting information indicating the test region.
